(19) United States
(12) Patent Application Publication
(10) Pub. No.: US 2022/0287647 A1
(43) Pub. Date: Sep. 15, 2022

(54) DISEASE CLASSIFICATION BY DEEP LEARNING MODELS

(71) Applicant: The University of Hong Kong, Hong Kong (CN)

(72) Inventors: Leung Ho Philip Yu, Hong Kong (CN); Wenming Cao, Hong Kong (CN); Chiu Sing Gilbert Lui, Hong Kong (CN); Wan Hang Keith Chiu, Hong Kong (CN); Man Fung Yuen, Hong Kong (CN); Wai Kay Walter Seto, Hong Kong (CN)

(21) Appl. No.: 17/692,861

(22) Filed: Mar. 11, 2022

Related U.S. Application Data
(60) Provisional application No. 63/160,377, filed on Mar. 12, 2021.

(51) Int. Cl. A61B 5/00; G06T 7/00; G06N 3/04

(52) U.S. Cl. CPC A61B 5/7264; G06T 7/0012; G06N 3/0454; A61B 5/743; G06T 2207/10081; G06T 2207/10116; G06T 2207/10088; G06T 2207/10132; G06T 2207/10104; G06T 2207/30056

(57) ABSTRACT

A computer-implemented system (CIS), based on the DenseNet model, for processing and/or analyzing computer tomography (CT) medical imaging input data is described. The CIS contains two or more dense blocks containing one or more modules. Within each dense block, output from preceding modules containing convolutional layers are transmitted to succeeding modules containing convolutional layers, via a gate that is controlled by a predefined or trainable threshold. The CIS also includes transition layers between the dense blocks, operably linked to pairs of consecutive dense blocks in the series configuration. The CIS can be used in a computer-implemented method for enhanced diagnoses of hepatocellular carcinoma, based analysis of one or more CT medical images.

### CROSS-REFERENCE TO RELATED APPLICATION

This application claims benefit of and priority to U.S. Provisional Application No. 63/160,377, filed on Mar. 12, 2021, which is hereby incorporated herein by reference in its entirety.

### FIELD OF THE INVENTION

This invention is generally related to processing and visualizing data, particularly a computer-implemented system/method for processing and visualizing images of liver tissue in clinical settings, to determine the presence of liver lesions that are indicative hepatocellular carcinoma.

### BACKGROUND OF THE INVENTION

Liver cancer is the fifth most common cancer in the world and is the third most common cause of cancer-related death (Bray, et al., CA: A Cancer Journal for Clinicians 2018, 68:394-424). Liver cancer has been one of the fatal cancers in the Asia-Pacific and accounted for 10.3% of all cancer deaths in Hong Kong in 2018 (Hong Kong Cancer Strategy by The Government of the Hong Kong Special Administrative Region, Published in July 2019, pages 1-100). Hepatocellular carcinoma (HCC) constitutes about 75-85% of primary liver cancer cases, and is one of the leading causes of mortality by cancer (Bray, et al., CA: A Cancer Journal for Clinicians 2018, 68:394-424). Consequently, early diagnosis and detection of HCC can help to improve its medical treatment.

The diagnosis of HCC typically does not require a liver biopsy, and is instead performed radiologically via cross-sectional imaging, e.g., computed tomography (CT) scan, particularly multiphase contrast CT scan, reported via the Liver Imaging Reporting and Data System (LI-RADS). A classical diagnosis of HCC is attained by the LI-RADS 5 category, defined as arterial phase enhancement followed by “washout” in the portal-venous or delayed phase (Marrero, et al., Hepatology 2018, 68(2):723-750). Nonetheless, the diagnostic categories of LI-RADS 2 to 4 represent varying risks of HCC, leading to repeated scans and a delay in diagnosis and treatment (van der Pol, et al., Gastroenterology 2019, 156(4):976-986).

Traditionally, clinicians have investigated slices of CT scan images visually. Accordingly, the diagnostic accuracy has heavily depended on the experience of radiologists. Thus, accurate diagnosis of liver lesions could be a challenging task and longer time could be spent to confirm the diagnosis. However, with rapid technological advances, especially in high-performance central processing units (CPUs) and graphics processing units (GPUs), artificial intelligence is increasingly being explored in medical diagnosis applications. For instance, attempts have been made to apply artificial intelligence, such as deep learning models that are essentially deep neural networks, to automate the procedure of diagnosis. These endeavors include attempts to diagnose liver cancer using CT images by classifying HCC or non-HCC. Yasada, et al., (Yasada, et al., Radiology 2018, 286(3):887-896), investigated the diagnostic effectiveness of convolutional neural networks to differentiate or classify liver masses ((A) hepatocellular carcinomas (HCC); (B) malignant liver tumors other than classic and early HCCs; (C) indeterminate masses or mass-like lesions and rare benign liver masses other than hemangiomas and cysts; (D) hemangiomas; (E) cysts). Ben-Cohen, et al., (Ben-Cohen, et al., Neurocomputing 2018, 275:1585-1594), proposed a liver metastases detection scheme that involves combining the global context using fully-convolutional networks (FCN) and local context using super-pixel sparse-based dictionary learning. Trivizakis, et al., (Trivizakis, et al., IEEE Journal of Biomedical and Health Informatics 2019, 23:923-930), used 3D-convolutional networks for tissue classification to distinguish primary and metastatic liver tumors in diffusion weighted magnetic resonance imaging data. Li, et al., (Li, et al., Computers in Biology and Medicine 2017, 84:156-167), investigated fusing extreme learning machine into fully-connected convolutional networks for nuclei grading of hepatocellular carcinoma. In addition, Li, et al., (Li, et al., Neurocomputing 2018, 312:9-26) further proposed a structure convolution extreme learning machine scheme for nucleus segmentation of HCC by fusing the information of case-based shape templates. Frid-Adar, et al., (Frid-Adar, et al., Neurocomputing 2018, 321:321-331), proposed a generative adversarial networks-based synthetic medical image augmentation framework to improve classification performances on CT liver images. Vivanti, et al., (Vivanti, et al., International Journal of Computer Assisted Radiology and Surgery 2017, 12:1945-1957; Vivanti, et al., Medical & Biological Engineering & Computing 2018, 56:1699-1713) proposed convolutional neural networks-based schemes to conduct tumor detection and delineation for longitudinal liver CT scans, respectively. Zhang, et al., (Zhang, et al., Liver tissue classification using an auto-context-based deep neural network with a multi-phase training framework. In: Bai W, Sanroma G, Wu G, Munsell B, Zhan Y, Coupe P. (eds) Patch-Based Techniques in Medical Imaging. Patch-MI 2018. Lecture Notes in Computer Science 2018, 11075:59-66), proposed a convolutional neural network-based scheme to classify different liver tissues for 3D magnetic resonance imaging (MRI) data of patients who are diagnosed as HCC, where the auto-context information capture module is integrated into a U-Net-shape architecture. Todoroki, et al., (Todoroki, et al., Detection of Liver Tumor Candidates from CT Images Using Deep Convolutional Neural Networks. In: Chen YW., Tanaka S., Howlett R., Jain L. (eds) Innovation in Medicine and Healthcare 2017. KES-InMed 2018 2017. 2018:71:140-145), proposed a two-stage convolutional network for classification of liver tumors, where in the first step livers in CT images were segmented using the algorithm developed by Dong, et al., (Dong, et al., Journal of Information Processing 2016, 24(2): 320-329; Dong, et al., Computers in Biology And Medicine 2015, 67:146-160), and in the second step deep convolutional neural network (DCNN) computed the probability of pixels in the segmented liver belonging to tumors. These computed probabilities were fed into fully connected layers to classify tumors. Lee, et al., (Lee, et al., Liver Lesion Detection from Weakly-labeled Multi-phase CT Volumes with a Grouped Single Shot MultiBox Detector. In: Frangi A., Schnabel J., Davatzikos C., Alberola-Lopez C., Fichtinger G. (eds) Medical Image Computing and Computer Assisted Intervention—MICCAI 2018. Lecture Notes in Computer Science, 2018, 11071:693-701), proposed single shot multi-box detector (SSD) for liver lesion detection, which incorporated group convolutions for feature maps and leveraged richer information of multi-phase CT images. Liang, et al., (Liang, et al., Combining Convolutional and Recurrent Neural Networks for Classification of Focal Liver Lesions in Multi-phase CT Images. In: Frangi A., Schnabel J., Davatzikos C., Alberola-Lopez C., Fichtinger G. (eds) Medical Image Computing and Computer Assisted Intervention—MICCAI 2018. Lecture Notes in Computer Science 2018, 11071:666-675), proposed ResGL-BDLSTM model to classify focal lesions of multi-phase CT liver images, in which the residual networks with global and local pathways and the bi-directional long short term memory were integrated. The performance of this model was evaluated on CT liver images which contained four types of lesions confirmed by pathologists, (i.e., cyst, hemangioma, follicular nodular hyperplasia, and HCC), which achieved 90.93% accuracy. As shown by these studies and their recencies, the efficient diagnoses of diseases, such as liver cancer (e.g., HCC) that involve analyses of medical images of tissue samples, is an unmet need and remains an area of active research. Accordingly, there remains a need in the area of medical diagnoses of diseases, such as liver cancers particularly HCC, involving analyses of images for more efficient diagnostic tools and/or reduction in the randomness of diagnosis, due to clinicians' experience and/or reduced performances of other diagnostic tools. Although these aforementioned deep network-based methods have provided satisfactory diagnostic performance for liver CT images, they suffer from some drawbacks: (1) it requires a large scale of liver CT images to train the models; and (2) the training of models requires advanced powerful computation resources, such as graphical processing units (GPUs), to support. Consequently, enhancing the diagnostic efficiencies and performances of these methods requires new and improved platforms.

Therefore, it is an object of the invention to provide improved diagnostic tools.

It is also an object of the invention to provide neural networks to improve the diagnosis of diseases.

It is a further object of the invention to provide neural networks to improve the diagnosis of cancers by analyzing images from cancerous tissue(s).

It is also an object of the invention to provide neural networks to improve the diagnosis of hepatocellular carcinoma by analyzing images from liver tissue for the presence of lesions associated with carcinoma.

### SUMMARY OF THE INVENTION

Computer-implemented systems (CIS) and computer-implemented methods (CIM) that are not limited to any particular hardware or operating system and that are useful for processing and/or analyzing medical imaging input data are described. The medical imaging data are preferably computer tomography (CT) scans. The CIS and/or CIM are preferably based on the DenseNet model. In some forms, the CIS and/or CIM contain:

(i) A first dense block, a second dense block, a third dense block, and a fourth dense block in a series configuration. Each dense block contains one or more modules, each containing a convolutional layer. Within each dense block, output from preceding modules containing convolutional layers are transmitted to succeeding modules containing convolutional layers within a dense block, via a gate that is controlled by a trainable threshold. Further, within each dense block, the original input into the dense block is also transmitted to the succeeding modules. Transmission of the original input to the succeeding modules, within each dense block, does not go through the gate. The convolutional layers contain a rectified linear unit activation function;

(ii) An initial max pooling layer operably linked to the first dense block. The initial max pooling layer has a stride size of 2;

(iii) An initial convolutional layer operably linked to the initial max pooling layer. The initial convolutional layer has a stride size of 2, and contains a rectified linear unit activation function;

(iv) Transition layers between the dense blocks, operably linked to pairs of consecutive dense blocks in the series configuration. The transition layers contain a convolutional layer and an average pooling layer. These convolutional layers and average pooling layers have a stride size of 1 and 2, respectively, and contain a rectified linear unit activation function; and/or

(v) A classification layer operably to the fourth dense block. The classification layer contains a terminal fully connect layer and a terminal average pooling layer. The fully connected layer contains a 4-D soft-max activation function.

A preferred CIS and/or CIM contains all of (i), (ii), (iii), (iv), and (v). Additional details of this preferred CIS and/or CIM are presented in Table 3 herein.

Also described are methods of using the CIS, including, but not limited to, diagnosing a disease or disorder of the liver, such as hepatocellular carcinoma.

### BRIEF DESCRIPTION OF THE DRAWINGS

FIG. 1 is a schematic of one of the three classification models used herein.

FIG. 2 is a schematic of one of the three classification models used herein.

FIGS. 3A, 3B, 3C, and 3D together are a schematic of one of the three classification models used herein.

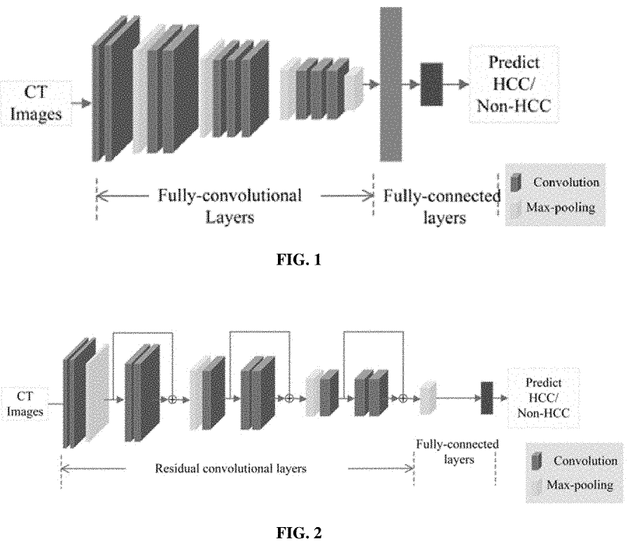

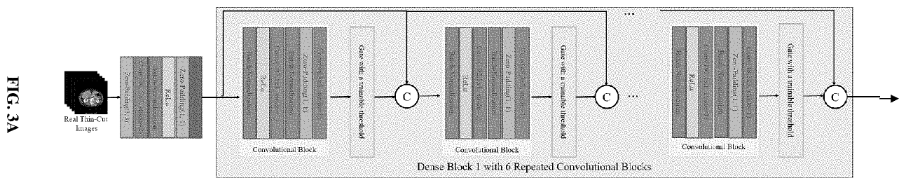

FIGS. 1, 2, and 3A-3D represent the fully convolutional networks model, deep residual network model, and densely connected convolutional network, respectively.

### DETAILED DESCRIPTION OF THE INVENTION

### I. Definitions

The term “activation function” describes a component of a neural network that may be used to bound neuron output, such as bounding between zero and one. Examples include soft-max, Rectified Linear Unit (“ReLU”), parametric rectified linear unit activation function (PReLu), or a sigmoid activation function.

The term “convolutional layer” describes a component in a neural network that transforms data (such as input data) in order to retrieve features from it. In this transformation, the data (such as an image) is convolved using one or more kernels (or one or more filters).

The term “dense block” describes a component in a neural network that contains layers, wherein output from a preceding layer are fed into succeeding layers. Preferably, within a dense block, feature map sizes are the same such that all the layers are easily connected.

The term “gate,” as used herein, refers to a component in a neural network that reduces the number of feature maps for a dense block by efficiently controlling information flow and depressing the effects of redundant information.

The term “pooling layer” refers to a component in a neural network, such as a DenseNet model, that performs down-sampling for feature compression. The “pooling layer” can be a “max pooling” layer or an “average pooling” layer. “Down-sampling” refers to the process of reducing the dimensions of input data compared to its full resolution, while simultaneously preserving the necessary input information for classification purposes. Typically, coarse representations of the input data (such as image) are generated.

The term “features,” as relates to neural networks, refers to variables or attributes in a data set. Generally, a subset of variables is picked that can be used as good predictors by a neural network model. They are independent variables that act like an input in the system. In the context of a neural network, the features would be the input layer, not what are known in the field as the “hidden layer nodes.”

The term “kernel” refers to a surface representation that can be used to represent a desired separation between two or more groups. The kernel is a parameterized representation of a surface in space. It can have many forms, including polynomial, in which the polynomial coefficients are parameters. A kernel can be visualized as a matrix (2D or 3D), with its height and width smaller than the dimensions of the data (such as input image) to be convolved. The kernel slides across the data (such as input image), and a dot product of the kernel and the input data (such as input image) are computed at every spatial position. The length by which the kernel slides is known as the “stride length.” Where more than one feature is to be extracted from the data (such as input image), multiple kernels can be used. In such a case, the size of all the kernels are preferably the same. The convolved features of the data (such as input image) are stacked one after the other to create an output so that the number of channels (or feature maps) is equal to the number of kernels used.

The term “segmentation” refers to the process of separating data into distinct groups. Typically, data in each group are similar of each other and different from data in other groups. In the context of images, segmentation involves identifying parts of the image and understanding to what object they belong. Segmentation can form the basis for performing object detection and classification. For an image of a biological organ, for example, segmentation can mean identifying the background, organ, parts of the organ, and instruction (where present).

### II. Computer-Implemented Systems and Methods

A classification network that is based on the DenseNet model is described. The DenseNet model allows for the direct transmission of information from the input and extracted features (such as extracted features of lesions) to the output layer in the network.

In traditional DenseNet models, within each dense block, all the outputs of preceding modules containing convolutional layers are input directly into succeeding modules containing convolutional layers within a dense block. In the DenseNet model described herein, within each dense block, output from preceding modules containing convolutional layers are transmitted to succeeding modules containing convolutional layers within a dense block, via a gate that is controlled by a trainable threshold. Preferably, within each dense block, the original input into the dense block is also transmitted to the succeeding modules. Preferably, transmission of the original input to the succeeding modules, within each dense block, does not go through the gate. In other words, each dense block is composed of multiple modules, referred to in FIG. 1 as convolutional blocks. For example, “dense block 1” includes six modules (or convolutional blocks). Within each dense block, the original input into the dense block is directly transmitted into succeeding modules (or succeeding convolutional blocks) bypassing the gate; while outputs from preceding modules (or preceding convolutional blocks) are transmitted to succeeding modules (or succeeding convolutional blocks). That is, the original input fed into all the succeeding modules (or succeeding convolutional blocks) within each dense block does not go through the gate. The described classification network incorporates this setting and simplifies the model architecture. The phrase “module,” and related terms, in the context of a dense block is used interchangeably with “convolutional block.” For example, “succeeding module” and “succeeding convolutional block” refer to the same component within dense block. Further, “preceding module” and “preceding convolutional block” refer to the same component within a dense block.

Further, the simplified architecture in the DenseNet-based model (i) reduces the number of feature maps for each dense block by controlling information flow efficiently and depressing the effects of redundant information, (ii) allows for increasing the number of dense blocks, i.e., the network depth, without the need to add too many parameters for tuning; (iii) simplifies the transition layer in dense blocks using convolutional and pooling layers alone, without any compression whose parameter requires careful parameterization, (iv) allows for the flexibility of classification data, such as images of liver lesions, and/or (v) reduces information loss, and subsequently improve classification performance.

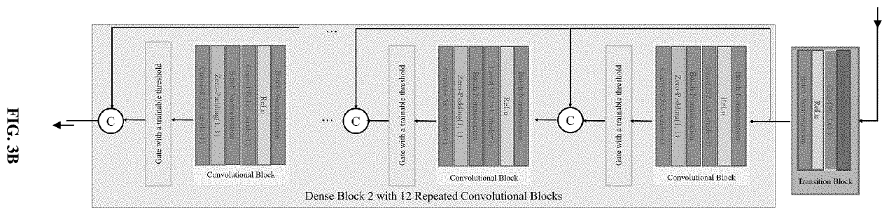

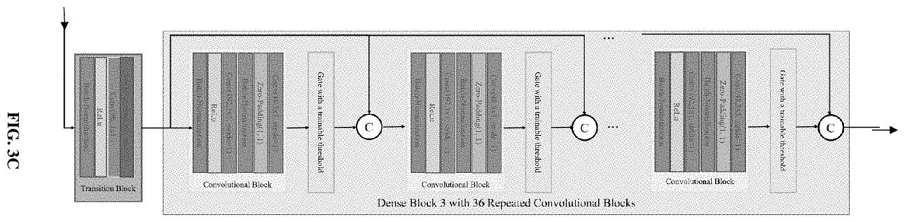

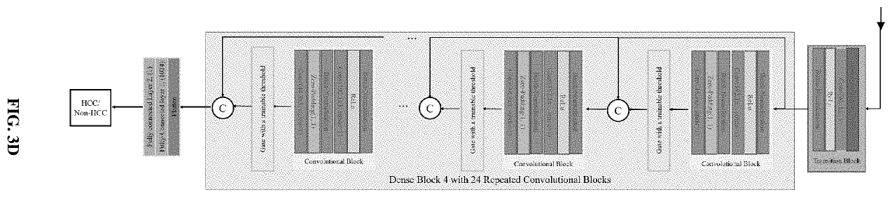

An overall, non-limiting architecture of the proposed DenseNet-based model is shown in FIGS. 3A-3D. Each dense block in the DenseNet-based model allows for the direct transmission of information from the input and extracted features (such as features of lesions) to the output in the network and this architecture can reduce the risk of diminishing and exploding gradients. The transition layers between two contiguous dense blocks can enhance the extracted features (such as features of lesions) in each preceding dense block for further feature extraction by the subsequent dense block. Further simplification of dense blocks by the number and dimension of feature maps can improve the adaptability of more diverse quality of cross-sectional images. Therefore, the described approach can combine the features of regions (such as lesion regions) to achieve accurate classification.

In the specific, non-limiting example of hepatocellular carcinoma (HCC), distinguishing HCC from non-HCC samples by the DenseNet based model on cross-sectional imaging can assist in the diagnosis of HCC accurately and efficiently. Experimental results show that the disclosed CIS and/or CIS can achieve better performance over the clinicians and other tested neural networks for at least the accuracy measure. Accordingly, the method provides a more efficient diagnosis and reduces the randomness, due to clinicians' experience. Consequently, the mortality risk of from diseases, such as HCC can be greatly reduced by appropriate medical treatment.

i. Computer-Implemented System

A computer-implemented system (CIS) that is not limited to any particular hardware or operating system is provided for processing and/or analyzing imaging and/or non-imaging input data is described. The CIS allows a user to make diagnoses or prognoses of a disease and/or disorder, based on output preferably displayed on a graphical user interface. A preferred disease and/or disorder includes hepatocellular carcinoma.

The CIS contains a first dense block and a second dense block. The first dense block, the second dense block, or both contain one or more succeeding modules that contain one or more convolutional layers. Within the first dense block, the second dense block, or both, output from a preceding module is transmitted to a convolutional layer in a succeeding module via a gate. Preferably, the gate has a trainable threshold. The trainable threshold can be fine-tuned by observing its effects on classification performance. It is used to choose informative features learnt by convolutional layers whose outputs are denoted in terms of feature maps with excessively redundant information. With this gate controlling mechanism, the number of feature maps transferred from the preceding convolutional layers to the following succeeding convolutional layers is reduced significantly. This cannot only suppress negative effects of redundant feature maps but also reduces the amount of network hyper-parameters. Preferably, the gate contains a correlation computation block and a controlling gating. The correlation computation block measures the Pearson correlation coefficients for feature maps learned by a given convolutional layer, and the controllable gating selects the top-25% (50% or 75%) discriminative features based on the obtained Pearson correlation coefficients. Thus, outputs of preceding convolutional layers are fed into the succeeding convolutional layer(s) along with the original input of each dense block. A non-limiting illustration is shown in FIGS. 3A-3D. In FIGS. 3A-3D, within each dense block, the component denoted “C” transmits the output from a preceding module along with the original image, to a succeeding module.

In some forms, within the first dense block, output from a preceding module is transmitted to a convolutional layer in a succeeding module via a gate having features as described above. Preferably, within the first dense block, the original input into the first dense block is also transmitted to the succeeding modules. Preferably, transmission of the original input to the succeeding modules, within the first dense block, does not involve the gate. That is, within the first dense block, the original input into the first dense block is transmitted into succeeding modules (or convolutional blocks) directly bypassing the gate, while transmission of outputs from a preceding module (or preceding convolutional block) to the succeeding modules (or succeeding convolutional blocks) involves the gate. In some forms, within the second dense block, output from a preceding module is transmitted to a convolutional layer in a succeeding module via a gate having features as described above. Preferably, within the second dense block, the original input into the second dense block is also transmitted to the succeeding modules. Preferably, transmission of the original input to the succeeding modules, within the second dense block, does not involve the gate. That is, within the second dense block, the original input into the second dense block is transmitted into succeeding modules (or convolutional blocks) directly bypassing the gate, while transmission of outputs from a preceding module (or preceding convolutional block) to the succeeding modules (or succeeding convolutional blocks) involves the gate. In some forms, within the first dense block and the second dense block, output from a preceding module is transmitted to a convolutional layer in a succeeding module via a gate with features as described above. Preferably, within the first dense block and the second dense block, the original input into the first dense block and the second dense block, respectively, is also transmitted to the succeeding modules within each of these dense blocks. Preferably, transmission of the original input in each respective block to the succeeding modules within each of these dense blocks, does not involve the gate. That is, within the first dense block and the second dense block, the original input into the first dense block and second dense block, respectively, is transmitted into succeeding modules (or convolutional blocks) within each dense block directly bypassing the gate, while transmission of outputs from a preceding module (or preceding convolutional block) to the succeeding modules (or succeeding convolutional blocks) within each of these dense blocks involves the gate. A non-limiting schematic is shown in FIGS. 3A-3D. In some forms, output from a preceding module is transmitted to all succeeding modules. In some forms, output is from a last convolutional layer in the preceding module. In some forms, output is transmitted to a first convolutional layer in a succeeding module(s).

Preferably, the first dense block and the second dense block are in a series configuration. In some forms, the first dense block has a higher number of kernels than the second dense block. In some forms, the kernels include 1×1 kernels, 3×3 kernels, or both. Preferably, the kernels include 1×1 kernels and 3×3 kernels.

In some forms, the CIS is as described above, except that the CIS further contains a transition layer operably linked to the first dense block and the second dense block. The transition layer the transition layer contains a convolutional layer (transition convolutional layer), a pooling layer (transition pooling layer), or both. Preferably, the transition layer contains a transition convolutional layer and a transition pooling layer.

In some forms, the transition convolutional layer contains one or more 1×1 kernels, preferably 96 kernels. In some forms, the transition convolutional layer has a stride size of one. Preferably, all the convolutional kernels in the transitional block have a size of 1×1 shown in Table 3 and the stride size is set to 1. The effects of stride sizes on performance of deep neural networks have been investigated (Karen Simonyan & Andrew Zisserman in ICLR 2015: Very deep convolutional networks for large-scale image recognition). Empirically, the proposed method can work well with other stride sizes.

In some forms, the transition convolutional layer contains an activation function layer selected from a rectified linear unit activation function (ReLu) layer, a parametric rectified linear unit activation function (PReLu) layer, or a sigmoid activation function layer. In some forms, the transition convolutional layer comprises a rectified linear unit activation function (ReLu) layer.

In some forms, the transition pooling layer contains an average pooling layer or a max pooling layer. In some forms, the transition pooling layer contains an average pooling layer. In some forms, the transition pooling layer contains one or more 2×2 kernels, preferably one kernel. In some forms, the transition pooling layer has a stride size of two. The stride size of the pooling layer in the transitional block of two is determined by the kernel size 2×2. Thus, the dimension of feature maps for succeeding dense blocks can be reduced without any overlapping.

In some forms, the CIS is as described above, except that the CIS further contains a third dense block. Preferably, the third dense block is operably linked to the second dense block via a first additional transition layer. In some forms, the third dense block is in series with the second dense block.

In some forms, the CIS is as described above, except that the CIS further contains a fourth dense block. Preferably, the fourth dense block is operably linked to the third dense block via a second additional transition layer. In some forms, the fourth dense block is in series with the third dense block.

In some forms, the third dense block, the fourth dense block, or both contain one or more succeeding modules containing one or more convolutional layers. Preferably, within the third dense block, the fourth dense block, or both, output from a preceding module is transmitted to a convolutional layer in a succeeding module via a gate with features as described above. Preferably, the gate in the third dense block or the fourth dense block independently has a trainable threshold. The trainable threshold can be fine-tuned by observing its effects on classification performance. It is used to choose informative features learnt by convolutional layers whose outputs are denoted in terms of feature maps with excessively redundant information. With this gate controlling mechanism, the number of feature maps transferred from the preceding convolutional layers to the following succeeding convolutional layers is reduced significantly. This cannot only suppress negative effects of redundant feature maps but also reduces the amount of network hyper-parameters. Preferably, the gate contains a correlation computation block and a controlling gating. The correlation computation block measures the Pearson correlation coefficients for feature maps learned by a given convolutional layer, and the controllable gating selects the top-25% (50% or 75%) discriminative features based on the obtained Pearson correlation coefficients. Thus, outputs of preceding convolutional layers are fed into the succeeding convolutional layer(s) along with the original input of each dense block. A non-limiting illustration is shown in FIGS. 3A-3D. In FIGS. 3A-3D, within each dense block, the component denoted “C” concatenates outputs from a preceding module (or convolutional block) through the gate with the original input. The concatenated result is then fed into a succeeding module (or succeeding convolutional block).

In some forms, within the third dense block, output from a preceding module is transmitted to a convolutional layer in a succeeding module via a gate with features as described above. Preferably, within the third dense block, the original input into the third dense block is also transmitted to the succeeding modules. Preferably, transmission of the original input to the succeeding modules, within the third dense block, does not involve the gate. That is, within the third dense block, the original input into the third dense block is transmitted into succeeding modules (or convolutional blocks) directly bypassing the gate, while transmission of outputs from a preceding module (or preceding convolutional block) to the succeeding modules (or succeeding convolutional blocks) involves the gate. In some forms, within the fourth dense block, output from a preceding module is transmitted to a convolutional layer in a succeeding module via a gate with features as described above. Preferably, within the fourth dense block, the original input into the fourth dense block is also transmitted to the succeeding modules. Preferably, transmission of the original input to the succeeding modules, within the fourth dense block, does not involve the gate. That is, within the fourth dense block, the original input into the fourth dense block is transmitted into succeeding modules (or convolutional blocks) directly bypassing the gate, while transmission of outputs from a preceding module (or preceding convolutional block) to the succeeding modules (or succeeding convolutional blocks) involves the gate. In some forms, within the third dense block and the fourth dense block, output from a preceding module is transmitted to a convolutional layer in a succeeding module via a gate with features as described above. Preferably, within the third dense block and the fourth dense block, the original input into the third dense block and the fourth dense block, respectively, is also transmitted to the succeeding modules within each of these dense blocks. Preferably, transmission of the original input in each respective block to the succeeding modules within each of these dense blocks, does not involve the gate. That is, within the third dense block and the fourth dense block, the original input into the third dense block and fourth dense block, respectively, is transmitted into succeeding modules (or convolutional blocks) within each dense block directly bypassing the gate, while transmission of outputs from a preceding module (or preceding convolutional block) to the succeeding modules (or succeeding convolutional blocks) within each of these dense blocks involves the gate. A non-limiting schematic is shown in FIGS. 3A-3D. In some forms, within the third dense block, the fourth dense block, or both the output from a preceding module is transmitted to all succeeding modules. In some forms, within the third dense block, the fourth dense block, or both, the output is from a last convolutional layer in the preceding module. In some forms, within the third dense block or the fourth dense block the output is transmitted to a first convolutional layer in the succeeding module.

In some forms, the third dense block has a higher number of kernels than the second dense block. In some forms, the third dense block has a lower number of kernels than the fourth dense block. In some forms, the kernels within the third dense block and the fourth dense block independently include 1×1 kernels, 3×3 kernels, or both. In some forms, the kernels within the third dense block and the fourth dense block include 1×1 kernels and 3×3 kernels.

As described above, preferably (i) the third dense block is operably linked to the second dense block via a first additional transition layer, and (ii) the fourth dense block is operably linked to the third dense block via a second additional transition layer.

In some forms, the first additional transition layer and the second additional transition layer independently contain a convolutional layer (first or second additional transition convolutional layer, i.e., first ATCL or second ATCL), a pooling layer (first or second additional transition pooling layer, i.e., first ATPL or second ATPL), or both.

In some forms, the first ATCL and second ATCL independently contain one or more 1×1 kernels, preferably 96 kernels. In some forms, the first ATCL and second ATCL have a stride size of one. In some forms, the first ATCL and second ATCL independently contain an activation function layer selected from a rectified linear unit activation function (ReLu) layer, a parametric rectified linear unit activation function (PReLu) layer, or a sigmoid activation function layer. In some forms, the first ATCL and second ATCL contain a rectified linear unit activation function (ReLu) layer.

In some forms, the first ATPL and second ATPL independently contain an average pooling layer or a max pooling layer. In some forms, the first ATPL and second ATPL independently contain an average pooling layer. In some forms, the first ATPL and second ATPL independently contain one or more 2×2 kernels, preferably one kernel. In some forms, the first ATPL and the second ATPL have a stride size of two.

In some forms, the CIS is as described above, except that the CIS further contains an initial pooling layer operably linked to the first dense block. In some forms, the initial pooling layer contains a max pooling layer or an average pooling layer, preferably a max pooling layer. In some forms, the initial pooling layer contains a 3×3 kernel, preferably with a stride size of 2.

In some forms, the CIS is as described above, except that the CIS further contains an initial convolutional layer. Preferably, the initial convolutional layer is operably linked to the initial pooling layer. In some forms, the initial convolutional layer contains one or more 7×7 kernels, such as 96 kernels, preferably with a stride size of 2.

In some forms, the CIS is as described above, except that the CIS further contains classification layer operably linked to a terminal dense block. For instance, where the CIS contains two dense blocks, such as the first dense block and the second dense block in series, the second dense block would be the terminal dense block and would be operably linked to the classification layer.

For instance, where the CIS contains three or four dense blocks in series, the third dense block or the fourth dense block would be the terminal dense block, respectively, and would be operably linked to the classification layer. A similar explanation follows where the CIS contains additional dense blocks beyond the non-limiting examples described herein.

In some forms, the classification layer comprises a fully connected layer, a terminal pooling layer, or preferably both. Preferably, the fully connected layer takes output from a previous dense block (preferably the terminal dense block), “flattens” the output and converts it into a vector (preferably a single vector) that can serve an input for the next stage, such as the terminal pooling layer. In some forms, the fully connected layer comprises a soft-max activation function, such as a 4-D soft-max activation function. In some forms, the terminal pooling layer contains an average pooling layer or a max pooling layer, preferably an average pooling layer. In some forms, the terminal pooling layer comprises one or more 7×7 kernels, such as one kernel.

ii. Computer-Implemented Method

Also described is a computer-implemented method (CIM) for analyzing data, which involves using any of the CISs described above. Preferably, the CIM involves visualizing on a graphical user interface, output from these CISs. Visualizing this output facilitates the diagnosis, prognosis, or both, of a disease or disorder in a subject. The disease or disorder includes, but is not limited to, tumors (such as liver, brain, or breast cancer, etc), cysts, joint abnormalities, abdominal diseases, liver diseases, kidney disorders, neuronal disorders, or lung disorders. A preferred disease or disorder is hepatocellular carcinoma.

In some forms, the data are images from one or more biological samples. The input imaging data are preferably from medical imaging applications, including, but not limited to, computed tomography (CT) scans, X-ray images, magnetic resonance images, ultrasound images, positron emission tomography images, magnetic resonance angiograms, and combinations thereof. Preferably, the images are internal body parts of a mammal. In some forms, the internal body parts are livers, brains, blood vessels, hearts, stomachs, prostates, testes, breasts, ovaries, kidneys, neurons, bones, or lungs. Preferred input imaging data are CT liver scans.

### III. Methods of Using

The described CIS or CIM can be utilized to analyze data. The CIS or CIM is one of general applicability and is not limited to imaging data from a patient population in a specific geographical region of the world. Preferably, the data are imaging data, such as medical imaging data obtained using well-known medical imaging tools such as computed tomography (CT) scans, X-ray images, magnetic resonance images, ultrasound images, positron emission tomography images, magnetic resonance angiograms, and combinations thereof. Within the context of medical imaging, the CIS or CIM can be employed in the diagnosis or prognosis of diseases or disorders.

The disclosed CISs and CIMs can be further understood through the following enumerated paragraphs or embodiments.

1. A computer-implemented system (CIS) containing a first dense block and a second dense block,

wherein the first dense block, the second dense block, or both contain one or more succeeding modules comprising one or more convolutional layers, and

wherein within the first dense block, the second dense block, or both, output from a preceding module is transmitted to a convolutional layer in a succeeding module via a gate.

2. The CIS of paragraph 1, wherein the gate has a predefined or trainable threshold.

3. The CIS of paragraph 1 or 2, wherein the gate contains a correlation computation block and a controlling gating.

4. The CIS of any one of paragraphs 1 to 3, wherein within the first dense block, output from a preceding module is transmitted to a convolutional layer in a succeeding module via a gate.

5. The CIS of any one of paragraphs 1 to 4, wherein within the second dense block, output from a preceding module is transmitted to a convolutional layer in a succeeding module via a gate.

6. The CIS of any one of paragraphs 1 to 5, wherein within the first dense block and the second dense block, output from a preceding module is transmitted to a convolutional layer in a succeeding module via a gate.

7. The CIS of any one of paragraphs 1 to 6, wherein the output from a preceding module is transmitted to all succeeding modules.

8. The CIS of any one of paragraphs 1 to 7, wherein the output is from a last convolutional layer in the preceding module.

9. The CIS of any one of paragraphs 1 to 8, wherein the output is transmitted to a first convolutional layer in the succeeding module.

10. The CIS of any one of paragraph 1 to 9, wherein within the first dense block, the second dense block, or both, an original input into the first dense block and the second dense block, respectively, is also transmitted to the succeeding modules within each of the dense blocks, preferably wherein transmission of the original input in each respective dense block to the succeeding modules within each of the dense block, does not involve the gate.

11. The CIS of paragraph 10, wherein transmission of the original input in each respective dense block to the succeeding modules within each of the dense blocks, does not involve the gate.

12. The CIS of any one of paragraphs 1 to 11, wherein the first dense block and the second dense block are in a series configuration.

13. The CIS of any one of paragraphs 1 to 12, wherein the first dense block has a higher number of kernels than the second dense block.

14. The CIS of paragraph 13, wherein the kernels contain 1×1 kernels, 3×3 kernels, or both.

15. The CIS of paragraph 13 or 14, wherein the kernels contain 1 xl kernels and 3×3 kernels.

16. The CIS of any one of paragraphs 1 to 15, further containing a transition layer operably linked to the first dense block and the second dense block.

17. The CIS of paragraph 16, wherein the transition layer contains a convolutional layer (transition convolutional layer), a pooling layer (transition pooling layer), or both.

18. The CIS of paragraph 16 or 17, wherein the transition layer contains a transition convolutional layer and a transition pooling layer.

19. The CIS of paragraph 17 or 18, wherein the transition convolutional layer contains one or more 1×1 kernels, preferably 96 kernels.

20. The CIS of any one of paragraphs 17 to 19, wherein the transition convolutional layer has a stride size of one.

21. The CIS of any one of paragraphs 17 to 20, wherein the transition convolutional layer contains an activation function layer selected from a rectified linear unit activation function (ReLu) layer, a parametric rectified linear unit activation function (PReLu) layer, or a sigmoid activation function layer.

22. The CIS of any one of paragraphs 17 to 21, wherein the transition convolutional layer contains a rectified linear unit activation function (ReLu) layer.

23. The CIS of any one of paragraphs 17 to 22, wherein the transition pooling layer contains an average pooling layer or a max pooling layer.

24. The CIS of any one of paragraphs 17 to 23, wherein the transition pooling layer contains an average pooling layer.

25. The CIS of any one of paragraphs 17 to 24, wherein the transition pooling layer contains one or more 2×2 kernels, preferably one kernel.

26. The CIS of any one of paragraphs 17 to 25, wherein the transition pooling layer has a stride size of two.

27. The CIS of any one of paragraphs 1 to 26, further containing a third dense block.

28. The CIS of paragraph 27, wherein the third dense block is operably linked to the second dense block via a first additional transition layer.

29. The CIS of paragraph 27 or 28, further containing a fourth dense block.

30. The CIS of paragraph 29, wherein the fourth dense block is operably linked to the third dense block via a second additional transition layer.

31. The CIS of any one of paragraphs 27 to 30, wherein the third dense block is in series with the second dense block.

32. The CIS of any one of paragraphs 29 to 31, wherein the fourth dense block is in series with the third dense block.

33. The CIS of any one of paragraphs 29 to 32, wherein the third dense block, the fourth dense block, or both comprise one or more succeeding modules containing one or more convolutional layers, and wherein within the third dense block, the fourth dense block, or both, output from a preceding module is transmitted to a convolutional layer in a succeeding module via a gate.

34. The CIS of paragraph 33, wherein the gate in the third dense block or the fourth dense block independently has a predefined or trainable threshold.

35. The CIS of paragraph 33 or 34, wherein the gate contains a correlation computation block and a controlling gating.

36. The CIS of any one of paragraphs 27 to 35, wherein within the third dense block, output from a preceding module is transmitted to a convolutional layer in a succeeding module via a gate.

37. The CIS of any one of paragraphs 29 to 36, wherein within the fourth dense block, output from a preceding module is transmitted to a convolutional layer in a succeeding module via a gate.

38. The CIS of any one of paragraphs 29 to 37, wherein within the third dense block and the fourth dense block, output from a preceding module is transmitted to a convolutional layer in a succeeding module via a gate.

39. The CIS of any one of paragraphs 29 to 38, wherein within the third dense block, the fourth dense block, or both the output from a preceding module is transmitted to all succeeding modules.

40. The CIS of any one of paragraphs 29 to 39, wherein within the third dense block, the fourth dense block, or both, the output is from a last convolutional layer in the preceding module.

41. The CIS of any one of paragraphs 29 to 40, wherein within the third dense block or the fourth dense block the output is transmitted to a first convolutional layer in the succeeding module.

42. The CIS of any one of paragraphs 27 to 41, wherein the third dense block has a higher number of kernels than the second dense block.

43. The CIS of any one of paragraphs 29 to 42, wherein the third dense block has a lower number of kernels than the fourth dense block.

44. The CIS of paragraph 43, wherein the kernels within the third dense block and the fourth dense block independently contain 1×1 kernels, 3×3 kernels, or both.

45. The CIS of paragraph 43 or 44, wherein the kernels within the third dense block and the fourth dense block contain 1 xl kernels and 3×3 kernels.

46. The CIS of any one of paragraphs 29 to 45, wherein within the third dense block, the fourth dense block, or both, an original input into the first dense block and the second dense block, respectively, is also transmitted to the succeeding modules within each of the dense blocks, preferably wherein transmission of the original input in each respective dense block to the succeeding modules within each of the dense block, does not involve the gate.

47. The CIS of paragraph 46, wherein transmission of the original input in each respective dense block to the succeeding modules within each of the dense blocks, does not involve the gate.

48. The CIS of any one of paragraphs 30 to 47, wherein the first additional transition layer and the second additional transition layer independently contain a convolutional layer (first or second additional transition convolutional layer, i.e., first ATCL or second ATCL), a pooling layer (first or second additional transition pooling layer, i.e., first ATPL or second ATPL), or both.

49. The CIS of paragraph 48, wherein the first ATCL and second ATCL independently contain one or more 1×1 kernels, preferably 96 kernels.

50. The CIS of paragraph 48 or 49, wherein the first ATCL and second ATCL have a stride size of one.

51. The CIS of any one of paragraphs 48 to 50, wherein the first ATCL and second ATCL independently contain an activation function layer selected from a rectified linear unit activation function (ReLu) layer, a parametric rectified linear unit activation function (PReLu) layer, or a sigmoid activation function layer.

52. The CIS of any one of paragraphs 48 to 51, wherein the first ATCL and second ATCL contain a rectified linear unit activation function (ReLu) layer.

53. The CIS of any one of paragraphs 48 to 52, wherein the first ATPL and second ATPL independently contain an average pooling layer or a max pooling layer.

54. The CIS of any one of paragraphs 48 to 53, wherein the first ATPL and second ATPL independently contain an average pooling layer.

55. The CIS of any one of paragraphs 48 to 54, wherein the first ATPL and second ATPL independently contain one or more 2×2 kernels, preferably one kernel.

56. The CIS of any one of paragraphs 48 to 55, wherein the first ATPL and the second ATPL have a stride size of two.

57. The CIS of any one of paragraphs 1 to 56, further containing an initial pooling layer operably linked to the first dense block.

58. The CIS of paragraph 57, wherein the initial pooling layer contains a max pooling layer or an average pooling layer, preferably a max pooling layer.

59. The CIS of paragraph 57 or 58, wherein the initial pooling layer contains a 3×3 kernel, preferably with a stride size of 2.

60. The CIS of any one of paragraphs 1 to 59, further containing an initial convolutional layer.

61. The CIS of paragraph 60, wherein the initial convolutional layer is operably linked to the initial pooling layer.

62. The CIS of paragraph 60 or 61, wherein the initial convolutional layer contains one or more 7×7 kernels, such as 96 kernels, preferably with a stride size of 2.

63. The CIS of any one of paragraphs 1 to 62, further contains classification layer operably linked to a terminal dense block.

64. The CIS of paragraph 63, wherein the classification layer contains a fully connected layer, a terminal pooling layer, or preferably both.

65. The CIS of paragraph 64, wherein the fully connected layer contains a soft-max activation function, such as a 4-D soft-max activation function.

66. The CIS of paragraph 64 or 65, wherein the terminal pooling layer contains an average pooling layer or a max pooling layer, preferably an average pooling layer.

67. The CIS of any one of paragraphs 64 to 66, wherein the terminal pooling layer contains one or more 7×7 kernels, such as one kernel.

68. A computer-implemented method (CIM) for analyzing data, the CIM involving visualizing on a graphical user interface, output from the CIS of any one of paragraphs 1 to 67.

69. The CIM of paragraph 68, wherein visualizing the output on the graphical user interface, provides a diagnosis, prognosis, or both, of a disease or disorder in a subject.

70. The CIM of paragraph 68 or 69, wherein the data are images of one or more biological samples.

71. The CIM of any one of paragraphs 68 to 70, wherein the data are images of internal body parts of a mammal.

72. The CIM of any one of paragraphs 68 to 71, wherein the data are images from livers, brains, blood vessels, hearts, stomachs, prostates, testes, breasts, ovaries, kidneys, neurons, bones, or lungs.

73. The CIM of any one of paragraphs 68 to 72, wherein the data are selected from the group consisting of computed tomography (CT) scans, X-ray images, magnetic resonance images, ultrasound images, positron emission tomography images, magnetic resonance angiograms, and combinations thereof.

74. The CIM of any one of paragraphs 68 to 73, wherein the data are CT liver scans.

75. The CIM of any one of paragraphs 69 to 74, wherein the disease or disorder includes tumors (such as liver, brain, or breast cancer, etc), cysts, joint abnormalities, abdominal diseases, liver diseases, kidney disorders, neuronal disorders, or lung disorders.

76. The CIM of any one of paragraphs 69 to 75, wherein the disease or disorder is hepatocellular carcinoma.

### EXAMPLES

### Example 1: Classification of Hepatocellular Carcinoma by Deep Learning Models

HCC is one the leading forms of cancer worldwide. This example verifies the clinical feasibility of three classification models with different neural architectures in distinguishing HCC from Non-HCC, to provide diagnostic assistance to clinicians.

One thousand two hundred and eighty-eight (1288) computed tomography (CT) liver scans along with the corresponding clinical information were retrieved from three different institutes in Hong Kong and Shenzhen. The recommendation of the American Association for the Study of Liver Diseases (AASLD) for HCC diagnosis was followed. The liver image reporting and data system (LI-RADS) classification in lesion classification was employed. All the liver lesions were manually contoured and labelled with diagnostic ground-truth. Three classification models were constructed based on different network architectures: fully convolutional network, residual network, and densely-connected convolutional network. The networks were then trained on the collected CT liver scans.

In total, 2551 lesions were retrieved from the 1288 CT liver scans. The mean size of lesions was 36.6±44.5 mm, with 826 lesions confirmed as HCC. The liver scans were split in a 7:3 ratio as the training and testing sets, and then used to train the three classification models. Among the classification models, the DenseNet-based model achieved the best performance, with a diagnostic accuracy of 97.14%, negative predictive value (NPV) 98.27%, positive predictive value (PPV) 95.45%, sensitivity 97.35%, and specificity 97.02%. ResNet-based model obtained the second-best performance, achieving a diagnostic accuracy of 95.49%, NPV 96.94%, PPV 92.31%, sensitivity 95.36%, and specificity 94.87%. FCN-based model achieved a diagnostic accuracy of 93.51%, NPV 95.63%, PPV 90.38%, sensitivity 93.38%, and specificity 93.36%. These were compared to the diagnostic accuracy of 89.09%, NPV 93.24%, PPV 83.44%, sensitivity 90.07%, and specificity 88.46% via LI-RADS.

In summary, the three deep network-based classification models performed better than the radiologists in the task of classifying HCC vs Non-HCC. Lastly, the visualization of feature maps learnt by convolutions in these three models on HCC and Non-HCC cases was illustrated and compared.

Materials and Methods

Acquisition of CT Images

1,288 patients underwent quadruple-phase Multi-detector Computed Tomography (MDCT) including the unenhanced phase, arterial phase, portal venous phase and equilibrium phase. As the data were obtained during the midst of the rapid development of MDCT technology, various MDCT scanners were used.

All CT scans were obtained in the craniocaudal direction. They are generated from one of the following sets of CT parameters:

(1) detector configuration, 128×0.625 mm; slice spacing, 7 mm; reconstruction interval, 5 mm and 1 mm; rotation speed, 0.5 s; tube voltage, 120; tube current, dynamic 175 to 350 mA/reference current 210 mA; and matrix size, 512×512.

(2) detector configuration, 8×1.25 mm, 16×1.5 mm and 64×0.625 mm; slice thickness, 2.5 mm, 3.0 mm and 3.0 mm; reconstruction interval, 2.5 mm, 3.0 mm and 3.0 mm; table speed, 13.5, 24.0 and 46.9 mm per rotation; 250, 200 and 175 mA effective current; rotation time, 0.5, 0.5 and 0.75 s; tube potential 120 kVp; and matrix size, 512×512.

The data used in this study were collected from the Pamela Youde Nethersole Eastern Hospital (PYNEH) in Hong Kong, the University of Hong Kong (HKU), and the University of Hong Kong-Shenzhen hospital (HKU_SZH) in Shenzhen. This study followed the recommendations of the AASLD for HCC diagnosis. LI-RADS classification in lesion categorization was also adopted. Diagnosis was validated by a clinical composite reference standard based on patients' outcomes over the subsequent 12 months. Each live lesion was manually contoured and labeled with diagnostic ground-truth. The data from PYNEH contained 455 cases, in which there were 69 HCC and 386 Non-HCC cases. The data from HKU contained 348 cases, in which there were 172 HCC and 176 Non-HCC cases. The data set from HKU_SZH contained 485 cases, in which there were 267 HCC and 218 Non-HCC cases. In total, the numbers of HCC and Non-HCC cases were 551 and 781, respectively. These cases were split in 7:3 ratio as the training set and testing set. The training set contained 354 HCC and 546 non-HCC cases. The test set contained 153 HCC and 235 non-HCC cases.

Table 1 shows the number of HCC and Non-HCC cases in these three data sets.

TABLE 1
The number of HCC and non-HCC cases in the training set
and testing sets in the data sets PYNEH, HKU and HKU_SZH.

| | Training | | Testing | |
|---|---|---|---|---|
| | # HCC | # Non-HCC | # HCC | # Non-HCC |
| PYNEH | 42 | 283 | 27 | 103 |
| HKU | 123 | 127 | 49 | 49 |
| HKU_SZH | 189 | 136 | 77 | 83 |
| Overall | 354 | 546 | 153 | 235 |

Table 2 summarizes the numbers of liver lesions of these data sets in the training and testing sets.

TABLE 2
The number of liver lesions in the training set and
testing sets in the data sets PYNEH, HKU and HKU_SZH.

| | Training | | Testing | |
|---|---|---|---|---|
| | # HCC | # Non-HCC | # HCC | # Non-HCC |
| | Lesions | Lesions | Lesions | Lesions |
| PYNEH | 67 | 564 | 38 | 213 |
| HKU | 289 | 233 | 58 | 86 |
| HKU_SZH | 288 | 362 | 86 | 267 |
| Overall | 644 | 1159 | 182 | 566 |

Classification Models

Three classification models were utilized to classify the lesion images of liver CT. These models included fully convolutional networks (FCN), deep residual network (ResNet), and densely connected convolutional network (DenseNet) as backbones for learning high-level features. An overview of the frameworks of these three classification models are shown in FIGS. 1, 2, and 3. Since the goal of classification models is to identify CT liver images as HCC or Non-HCC, i.e., binary classification problem, the cross-entropy loss function is chosen as the optimization function to train weights of these deep network models.

Details of the architectures of the three classification models are shown in Table 3, and further described below.

TABLE 3
Details of the architectures of the FCN-based, ResNet-based, and DenseNet-based
models utilized in this study.

| FCN-based Model | | ResNet-based Model | | DenseNet-based Model | |
|---|---|---|---|---|---|
| Layer | | Layer | | Layer | |
| Name | Input | Name | Input | Name | Input |
| block1_conv | $[ \begin{matrix}3 \times 3 , & 64 \\ 3 \times 3 , & 64\end{matrix} ]$ | Conv1 | [7 × 7, 64], stride = 2 | conv | [7 × 7, 96], stride = 2 |
| block1_pool | 2 × 2 max | Pooling_1 | 3 × 3 max pool, | Pooling | 3 × 3 max |
| | pool, stride = 2 | | stride = 2 | | pool, stride |
| | | | | | = 2 |
| block2_conv | $[ \begin{matrix}3 \times 3 , & 128 \\ 3 \times 3 , & 128\end{matrix} ]$ | Conv2_x | $[ \begin{matrix}3 \times 3 , & 64 \\ 3 \times 3 , & 64 \\ 3 \times 3 , & 256\end{matrix} ] \times 3$ | DenseBlock1_x | $[ \begin{matrix}1 \times 1 \\ 3 \times 3\end{matrix} ] \times 6$ |
| block2_pool | 2 × 2 max pool, stride = 2 | Conv3_x | $[ \begin{matrix}3 \times 3 , & 128 \\ 3 \times 3 , & 128 \\ 3 \times 3 , & 512\end{matrix} ] \times 4$ | Transit1_x | [1 × 1, 96], stride = 1 2 × 2 avg pool, stride = 2 |
| block3_conv | $[ \begin{matrix}3 \times 3 , & 256 \\ 3 \times 3 , & 256 \\ 3 \times 3 , & 256\end{matrix} ]$ | Conv4_x | $[ \begin{matrix}3 \times 3 , & 256 \\ 3 \times 3 , & 256 \\ 3 \times 3 , & 1024\end{matrix} ] \times 6$ | DenseBlock2_x | $[ \begin{matrix}1 \times 1 \\ 3 \times 3\end{matrix} ] \times 12$ |
| block3_pool | 2 × 2 max pool, stride = 2 | Conv5_x | $[ \begin{matrix}3 \times 3 , & 512 \\ 3 \times 3 , & 512 \\ 3 \times 3 , & 2048\end{matrix} ] \times 6$ | Transit2_x | [1 × 1, 96], stride = 1 2 × 2 avg pool, stride = 2 |
| block4_conv | $[ \begin{matrix}3 \times 3 , & 512 \\ 3 \times 3 , & 512 \\ 3 \times 3 , & 512\end{matrix} ]$ | FC | 4-D, Soft-max | DenseBlock3_x | $[ \begin{matrix}1 \times 1 \\ 3 \times 3\end{matrix} ] \times 36$ |
| block4_pool | 2 × 2 max | | | Transit3_x | [1 × 1, 96], |
| | pool, stride = 2 | | | | stride = 1 |
| | | | | | 2 × 2 avg |
| | | | | | pool, stride |
| | | | | | = 2 |
| block5_conv | [7 × 7, 4096] | | | DenseBlock4_x | $[ \begin{matrix}1 \times 1 \\ 3 \times 3\end{matrix} ] \times 24$ |
| FC | 4-D, Soft-max | | | Pooling | 7 × 7 avg |
| | | | | | pool |
| | | | | FC | 4-D, Soft-max |

i. FCN-Based Classification Model

The FCN-based model (Table 3) is composed of five blocks. The first block includes two blocks: block1_conv and block1_pool, in which block1_conv has two consecutive convolutional layers with 64 3×3 kernels, and block1_pool is a 2×2 max-pooling layer with stride=2. The second block also includes two blocks: block2_conv and block2_pool, in which block2_conv has two consecutive convolutional layers with 128 3×3 kernels, and block2_pool is a 2×2 max-pooling layer with stride=2.

Similarly, the third block contains two blocks: block3 conv and block4 pool, in which block3 conv has three consecutive convolutional layers with 256 3×3 kernels, and block3 pool is a 2×2 max-pooling layer with stride=2. The fourth block also includes two blocks: block4 conv and block4 pool, in which block4 conv has three consecutive convolutional layers with 512 3×3 kernels, and block4 pool is a 2×2 max-pooling layer with stride=2.

The fifth block is composed of a convolutional layer with 4096 7×7 kernels and a fully-connected layer.

The activation function in all the convolutional layers is rectified linear unit activation function (RELU), while the activation function in the fully-connected layer is Soft-max.

ii. ResNet-Based Classification Model

ResNet-based classification model (Table 3), is composed of 59 layers, among which there are 58 convolutional layers and one fully-connected layer. Conv1 is a convolutional layer with 64 7×7 kernels and stride=2. Conv2_x denotes three groups of three consecutive convolutional layers. Conv3_x, Conv4_x, and Conv5_x have four, six and six groups of three consecutive convolutional layers with different numbers of 3×3 kernels, respectively, as shown in Table 3. Stacked on the last convolutional layer, a fully-connected layer, adopted to classify the learned high-level features into two classes.

All the convolutional layers use RELU as the activation function, while the fully-connected layer uses Soft-max as the activation function.

iii. DenseNet-Based Classification Model

The DenseNet-based classification model (Table 3), includes four dense blocks, namely, DenseBlock1_x, DenseBlock2_x, . . . , DenseBlock4_x. The dense blocks are connected via transition blocks, i.e., Transit1_x, Transit2_x, . . . , and Transit3_x. Each dense block is composed of several consecutive modules. The dense blocks are arranged consecutively and contain growing numbers of 1×1 and 3×3 kernels, except in some cases where the last dense block in the series contains a lower number of 1×1 and 3×3 kernels compared with its immediately preceding dense block. For example, DenseBlock1_x has six modules, each of which contains two convolutional layers. Each transition block, which is used to change sizes of feature maps, is composed of a convolutional layer and a pooling layer. The pooling layers in the transit blocks have a stride=2.

In the above three classification models, all the convolutional layers use RELU as the activation function, while the fully-connected layer uses Soft-max as the activation function.

Results

The performances of the above three deep networks in terms of classifying images were evaluated, which include quantitative and qualitative comparisons. For quantitative comparisons, the accuracy, specificity, sensitivity, PPV, and NPV were adopted as evaluation metrics. For qualitative comparisons, illustrations of feature maps learned by convolutions were generated and compared with annotated masks of liver lesions. The technique of Grad-Cam (Selvaraju, et al., The IEEE International Conference on Computer Vision (ICCV) 2017, 618-626) was implemented to visualize the classification results, i.e., the estimated location of liver lesions.

i. Quantitative Comparisons

Table 4 shows a quantitative comparison of the performances of the above-described deep networks.

TABLE 4
Quantitative comparisons among FCN-based, ResNet-based and
DenseNet-based classification models on PYNEH, HKU, HKU_SZH.

| | | FCN-based Model | | | ResNet-based Model | | | DenseNet-based Model | | |
|---|---|---|---|---|---|---|---|---|---|---|
| | | Predicted | | | Predicted | | | Predicted | | |
| | | | Non- | | | Non- | | | Non- | |
| | | HCC | HCC | Metric | HCC | HCC | Metric | HCC | HCC | Metric |
| The | HCC | 141 | 10 | Sensitivity = | 144 | 7 | Sensitivity = | 147 | 4 | Sensitivity = |
| Ground- | | | | 93.38% | | | 95.36% | | | 97.35% |
| Truth | Non- | 15 | 219 | Specificity = | 12 | 222 | Specificity = | 7 | 227 | Specificity = |
| | HCC | | | 93.36% | | | 94.87% | | | 97.02% |
| | Metric | PPV = | NPV = | Accuracy = | PPV = | NPV = | Accuracy = | PPV = | NPV = | Accuracy = |
| | | 90.38% | 95.63% | 93.51% | 92.31% | 96.94% | 95.49% | 95.45% | 98.27% | 97.14% |

It can be observed that DenseNet-based classification model achieved the best accuracy of 97.14%, compared to FCN-based and ResNet-based models. Specifically, it is 3.63% and 1.65% better than those of FCN-based and ResNet-based models, respectively. In addition, DenseNet-based classification models achieved specificity of 97.02%, which exceeds those of FCN-based and ResNet-based models by 3.66% and 2.15%, respectively. Meanwhile, DenseNet-based model performed best in terms of positive predictive value (PPV), which is 1.99% ahead of Resnet-based model and 3.97% ahead of FCN-based model.

Next, the performances of the DenseNet-based classification model and radiologists using the LI-RADS method were compared. The results are shown in Table 5.

TABLE 5
The comparisons between DenseNet-based classification model and
radiologists using the LI-RADS method on PYNEH, HKU and HKU_SZH.

| | | DenseNet-based Model | | | Radiologists | | |
|---|---|---|---|---|---|---|---|
| | | Predicted | | | Predicted | | |
| | | HCC | Non-HCC | Metric | HCC | Non-HCC | Metric |
| The | HCC | 147 | 4 | Sensitivity = | 136 | 15 | Sensitivity = |
| Ground- | | | | 97.35% | | | 90.07% |
| Truth | Non-HCC | 7 | 227 | Specificity = | 27 | 207 | Specificity = |
| | | | | 97.01% | | | 88.46% |
| | Metric | PPV = | NPV = | Accuracy = | PPV = | NPV = | Accuracy = |
| | | 95.45% | 98.27% | 97.14% | 83.44% | 93.24% | 89.09% |

As can be seen, DenseNet-based model has outperformed radiologists in terms of all the evaluation metrics. Specifically, DenseNet-based model improved the diagnostic accuracy, NPV, PPV, sensitivity, and specificity compared to radiologists whose respective values were 89.09%, 93.24%, 83.44%, 90.07%, and 88.46%, respectively. Evaluating the data in Table 4 and Table 5, shows that the DenseNet-based model achieved the best performance, followed by ResNet-based model (the second-best), and FCN-based model. All the three classification models outperformed the radiologists.

ii. Qualitative Comparisons

To explore differences of HCC against Non-HCC cases, visualizations of feature maps learned by the three classification models are generated. The visualization of feature maps when inputting into three cases with different lesion sizes, denoted as large, medium, and small were compared. There are 73 pixels, 64 pixels and 35 pixels in the longest diameter for three HCC lesions, respectively. The images showed that, all the red zones of heat maps of features learned by the three deep network-based models have strong correlations with lesions of liver. In other words, when red zones appear in a heat map of features, there is a high probability that the CT image has HCC lesions.

The data show that features learned by DenseNet-based classification model are more beneficial to classifying small-size or medium-size lesions, compared with ResNet-based and FCN-based models. While in the case of large-size lesions, red zones of heat maps in the feature maps learned by FCN-based model tend to become smaller, compared to ResNet-based and DenseNet-based model. This leads to a worse diagnosis for large-size HCC lesion, which is undesirable. An interesting observation is that although the learned feature maps in ResNet-based model can detect the appearing of small-size HCC lesion, it tends to locate the lesion with a larger deviation, compared to FCN-based and DenseNet-based models. As a result, the DenseNet-based classification model achieved better performance.

In contrast, when the input CT liver images are identified as non-HCC, there are no red hot zones in the feature maps learned by convolutional layers of the three classification models, regardless of the lesion sizes. In other words, the appearance of red hot zones in the learnt feature maps is considered as an indicator of HCC, which can also locate the position of HCC lesions and reduce the diagnostic time.

By comparing the performance of radiologists with those of three classification models based on different network architectures, the following have been observed:

(1) all the classification models based on different network architectures outperformed radiologists;

(2) DenseNet-based model achieved the best performance, compared to FCN-based and ResNet-based models;

(3) by presenting the visualizations of feature maps learned by three models when inputting CT liver images with HCC lesions and non-HCC lesions, the advantages of DenseNet-based model over FCN-based and ResNet-based models were analyzed.

Those skilled in the art will recognize, or be able to ascertain using no more than routine experimentation, many equivalents to the specific embodiments of the invention described herein. Such equivalents are intended to be encompassed by the following claims.

We claim:

1. A computer-implemented system (CIS) comprising a first dense block and a second dense block,
wherein the first dense block, the second dense block, or both comprise one or more succeeding modules comprising one or more convolutional layers, and
wherein within the first dense block, the second dense block, or both, output from a preceding module is transmitted to a convolutional layer in a succeeding module via a gate.

2. The CIS of claim 1, wherein the gate has a trainable threshold.

3. The CIS of claim 1, wherein the gate comprises a correlation computation block and a controlling gating.

4. The CIS of claim 1, wherein the output is from a last convolutional layer in the preceding module.

5. The CIS of claim 1, wherein the output is transmitted to a first convolutional layer in the succeeding module.

6. The CIS of claim 1, wherein within the first dense block, the second dense block, or both, an original input into the first dense block and the second dense block, respectively, is also transmitted to the succeeding modules within each of the dense blocks.

7. The CIS of claim 1, wherein the first dense block has a higher number of kernels than the second dense block.

8. The CIS of claim 1, further comprising a transition layer operably linked to the first dense block and the second dense block.

9. The CIS of claim 8, wherein the transition layer comprises a convolutional layer, a pooling layer, or both.

10. The CIS of claim 9, wherein the transition convolutional layer comprises an activation function layer selected from a rectified linear unit activation function (ReLu) layer, a parametric rectified linear unit activation function (PReLu) layer, or a sigmoid activation function layer.

11. The CIS of claim 9, wherein the transition convolutional layer comprises a rectified linear unit activation function (ReLu) layer.

12. The CIS of claim 9, wherein the transition pooling layer comprises an average pooling layer or a max pooling layer.

13. The CIS of claim 1, further comprising an initial pooling layer operably linked to the first dense block.

14. The CIS of claim 13, wherein the initial pooling layer comprises a max pooling layer or an average pooling layer.

15. The CIS of claim 1, further comprising an initial convolutional layer.

16. The CIS of claim 15, wherein the initial convolutional layer is operably linked to the initial pooling layer.

17. The CIS of claim 1, further comprising classification layer operably linked to a terminal dense block.

18. The CIS of claim 17, wherein the classification layer comprises a fully connected layer, a terminal pooling layer, or both.

19. The CIS of claim 18, wherein the fully connected layer comprises a soft-max activation function.

20. The CIS of claim 18, wherein the terminal pooling layer comprises an average pooling layer or a max pooling layer.

21. A computer-implemented method (CIM) for analyzing data, the CIM comprising visualizing on a graphical user interface, output from the CIS of claim 1.

22. The CIM of claim 21, wherein visualizing the output on the graphical user interface, provides a diagnosis, prognosis, or both, of a disease or disorder in a subject.

23. The CIM of claim 21, wherein the data are images of one or more biological samples.

24. The CIM of claim 21, wherein the data are images of internal body parts of a mammal.

25. The CIM of claim 21, wherein the data are selected from the group consisting of computed tomography (CT) scans, X-ray images, magnetic resonance images, ultrasound images, positron emission tomography images, magnetic resonance angiograms, and combinations thereof.

26. The CIM claim 21, wherein the data are CT liver scans.

27. The CIM of claim 22, wherein the disease or disorder is hepatocellular carcinoma.
